(19) United States
(12) Patent Application Publication
(10) Pub. No.: US 2017/0266466 A1
(43) Pub. Date: Sep. 21, 2017

(54) AGENTS AND METHODS FOR THE TEMPORARY SHAPING OF KERATIN-CONTAINING FIBERS

(71) Applicant: Henkel AG & Co. KGaA, Duesseldorf (DE)

(72) Inventors: Thomas Foerster, Duesseldorf (DE); Rolf Bayersdoerfer, Hamburg (DE); Thorsten Knappe, Schenefeld (DE)

(73) Assignee: Henkel AG & Co. KGaA, Duesseldorf (DE)

(21) Appl. No.: 15/532,160

(22) PCT Filed: Nov. 2, 2015
(86) PCT No.: PCT/EP2015/075377

§ 371 Date: Jun. 1, 2017

(30) Foreign Application Priority Data
Dec. 10, 2014 (DE) 10 2014 225 438.8

(51) Int. Cl. A61Q 5/06; A61K 8/42; A61K 8/02; A61K 8/88; A61K 8/34; A61K 8/25; A61K 8/81; A61K 8/97

(52) U.S. Cl. CPC A61Q 5/06; A61K 8/8123; A61K 8/42; A61K 8/97; A61K 8/88; A61K 8/345; A61K 8/25; A61K 8/027; A61K 2800/54; A61K 2800/20; A61K 2800/87

(57) ABSTRACT

The invention relates to a cosmetic product comprising a) a cosmetic preparation containing, in relation to its total weight, a1) from about 60 to about 98 wt % of at least one polar solvent; a2) from about 0.1 to about 20 wt % of at least one copolymer from the monomers i) n-tert-butylacrylamide, ii) acrylic acid, iii) ethyl acrylate, iv) and optionally other monomers; b) a device for flash evaporation of the cosmetic preparation a), and to a method using corresponding products and to the use of the cosmetic preparation a) as process material in a device for flash evaporation.

### CROSS-REFERENCE TO RELATED APPLICATION

This application is a U.S. National-Stage entry under 35 U.S.C. §371 based on International Application No. PCT/EP2015/075377, filed Nov. 2, 2015 which was published under PCT Article 21(2) and which claims priority to German Application No. 102014225438.8, filed Dec. 10, 2014, which are all hereby incorporated in their entirety by reference.

### TECHNICAL FIELD

The application relates to the technical field of the temporary shaping of keratin-containing fibers, in particular human hair. Specific cosmetic formulations for the hair, which are suitable for application to keratin-containing fibers by employing a flash evaporation process, form the subject matter of the application. Also forming the subject matter of the present application are the use of these cosmetic formulations for the hair in devices for flash evaporation, as well as methods for the temporary shaping of keratin-containing fibers.

### BACKGROUND

An attractive hairstyle is nowadays generally regarded as an indispensable part of a well-groomed appearance. Based on current fashion trends, hairstyles which are considered to be chic are often those which on many hair types can be formed and/or maintained for a relatively long period of time of up to several days only by using active setting ingredients. Hair treatment agents which serve for permanently or temporarily shaping the hair therefore play an important role. While in the case of permanent shaping the chemical structure of the keratin-containing fibers is modified by reduction and oxidation, such modifications to the chemical structure do not take place in the case of temporary shaping. Corresponding temporary shaping agents usually contain synthetic polymers and/or waxes as the active setting ingredient.

The most important property of an agent for the temporary shaping of keratin-containing fibers, hereinafter also called styling agents, lies in giving the greatest possible hold to the treated fibers in the newly modeled shape, that is to say a shape impressed on the fibers. If the keratin-containing fibers are human hair, this is also referred to as a strong hairstyle hold or a high degree of hold of the styling agent. The hairstyle hold is substantially determined by the type and amount of the active setting ingredients used, but the other constituents of the styling agent and also the mode of application can also have an effect.

In the field of the temporary shaping of keratin-containing fibers, in particular the spray application of suitable cosmetic preparations has great significance, wherein the preparations are usually applied as pump sprays or aerosol sprays. To this end, the cosmetic preparations are usually packaged in a dispensing device, from which they are sprayed via a valve either by employing mechanical force or with the aid of a propellant. Both methods have obvious disadvantages. While pump sprays are generally not suitable for a sustained and even spray application of cosmetic hair preparations, aerosol sprays are based on the use of propellants or propellant gases, which on the one hand have no cosmetic effect and on the other hand can pose a risk to consumers if not handled correctly

Against this background, there is a need for alternative ways of atomizing cosmetic hair preparations. Flash evaporation has proven to be advantageous as one such alternative spraying method. In this method, which is described for example in international patent application WO 2001/83071 A1 (Henkel), a liquid or paste-like solvent-containing composition is heated in a closed chamber to a temperature above the boiling point of the solvent, thereby generating an overpressure in the composition. When the pressure is released (throttled), the liquid evaporates and can then be atomized for example by employing a suitable nozzle.

Therefore, although flash evaporation is suitable in principle for the spray application of cosmetic hair preparations, at the same time it is not possible to atomize every cosmetic hair preparation by employing a flash evaporation method. This is due on the one hand to the heating of the cosmetic preparation that is necessary for the flash evaporation, and on the other hand to the specifics of the spray mist produced by flash evaporation, for example the droplet size and droplet density produced in the spray mist.

### BRIEF SUMMARY

A cosmetic product is provided herein. The cosmetic product includes a cosmetic preparation including, in relation to its total weight, from about 60 to about 98 wt % of at least one polar solvent and from about 0.1 to about 20 wt % of at least one copolymer. The at least one copolymer is from the monomers N-tert-butylacrylamide, acrylic acid, ethyl acrylate, and optionally other monomers. The cosmetic product further includes a device for flash evaporation of the cosmetic preparation.

A cosmetic preparation is also provided herein. The cosmetic preparation includes, in relation to its total weight, from about 60 to about 98 wt % of at least one polar solvent and from about 0.1 to about 20 wt % of at least one copolymer. The at least one copolymer is from the monomers N-tert-butylacrylamide, acrylic acid, ethyl acrylate, and optionally other monomers. The cosmetic preparation is utilized as a material in a device for flash evaporation.

A method for the temporary shaping of keratin-containing fibers is also provided herein. The method includes applying a cosmetic preparation to the keratin-containing fibers by employing a device for flash evaporation. The cosmetic preparation includes, in relation to its total weight, from about 60 to about 98 wt % of at least one polar solvent and from about 0.1 to about 20 wt % of at least one copolymer. The at least one copolymer is from the monomers N-tert-butylacrylamide, acrylic acid, ethyl acrylate, and optionally other monomers.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The problem addressed by the present disclosure was therefore that of providing specific cosmetic hair preparations for the temporary shaping of keratin-containing fibers, which on account of their chemical and physical properties are suitable for targeted spray application by employing a device for flash evaporation. The preparations are also intended to be suitable for achieving a high degree of hold, in particular a high long-term hold, and a high-volume effect after application by employing a flash evaporation method. It has been found that, among the many known polymer preparations having a cosmetic effect on the hair, solvent-containing preparations based on a copolymer from the monomers N-tert-butylacrylamide, acrylic acid and ethyl acrylate are particularly suitable for solving said problem.

A first subject matter of the present disclosure is thus a cosmetic product comprising:

a cosmetic preparation containing, in relation to its total weight,

a1) from about 60 to about 98 wt % of at least one polar solvent;
a2) from about 0.1 to about 20 wt % of at least one copolymer from the monomers
i) N-tert-butylacrylamide
ii) acrylic acid
iii) ethyl acrylate
iv) and optionally other monomers;

a device for flash evaporation of the cosmetic preparation a).

The cosmetic preparation a) is preferably liquid. The cosmetic preparation a) may exist as a solution or dispersion, for example as an emulsion or suspension. Preferred cosmetic preparations a) are in the form of a solution or a suspension.

The cosmetic preparation as contemplated herein contains, as a first essential constituent, from about 60 to about 98 wt % of at least one polar solvent a1). Preferred cosmetic products are characterized in that the proportion by weight of the polar solvent a1) in relation to the total weight of the cosmetic preparation a) is from about 70 to about 98 wt %, preferably from about 80 to about 98 wt %. Such agents are characterized by a good cosmetic effect while at the same time being easy to apply.

In order to improve the application properties of cosmetic preparations as contemplated herein while at the same time minimizing the thermal stress of any active ingredients or auxiliaries in the course of the flash evaporation process, it has proven to be advantageous to use polar solvents a1) which have a boiling point (20° C. and 1013 mbar) between about 50 and about 110° C., preferably between about 70 and about 105° C. Ethanol, isopropanol and water have proven to be particularly suitable, and for this reason they are preferred as polar solvents a1).

Particularly preferred polar solvents a1) or solvent systems are
polar solvents a1) comprising more than 70 wt %, preferably more than 80 wt % and in particular more than 90 wt % ethanol, in each case based on the total weight of the polar solvent;
polar solvents a1) comprising, in each case based on the total weight of the polar solvent, more than 80 wt %, preferably more than 90 wt % and in particular more than 95 wt % of a mixture of ethanol and water, wherein the weight ratio of ethanol to water is preferably from about 1000:1 to about 200:1, preferably from about 1000:1 to about 500:1;
polar solvents a1) comprising more than 80 wt %, preferably more than 88 wt % and in particular more than 92 wt % water, in each case based on the total weight of the polar solvent.

Very particularly preferred cosmetic preparations are characterized in that the proportion of volatile constituents is at most 55 wt %. The group of said volatile constituents includes, inter alia, also the polar solvent ethanol. Very particularly preferred cosmetic preparations are thus in other words characterized in that the proportion by weight of ethanol in the total weight of the cosmetic preparation is at most 55 wt %, preferably from about 10 to about 55 wt %, particularly preferably from about 25 to about 55 wt % and in particular from about 40 to about 55 wt %.

Another preferred subject matter of the present disclosure is therefore a cosmetic product comprising

a) a cosmetic preparation containing, in relation to its total weight,
a1) from about 60 to about 98 wt % of at least one polar solvent, wherein the proportion by weight of ethanol in the total weight of the cosmetic preparation is at most 55 wt %, preferably from about 10 to about 55 wt %, particularly preferably from about 25 to about 55 wt % and in particular from about 40 to about 55 wt %;
a2) from about 0.1 to about 20 wt % of at least one copolymer from the monomers
i) N-tert-butylacrylamide
ii) acrylic acid
iii) ethyl acrylate
iv) and optionally other monomers;
b) a device for flash evaporation of the cosmetic preparation a), comprising
b1) a container b1) which can be closed and opened by employing a valve,
b2) a heating device which makes it possible to heat a cosmetic preparation located in the container b1),
b3) a nozzle b3) which enables atomization of the cosmetic preparation a).

A preferred combination of polar solvents a1) comprises water and ethanol, wherein
the proportion by weight of water and ethanol in the total weight of the polar solvent a1) is preferably at least about 60 wt %, more preferably at least about 80 wt %, particularly preferably at least about 90 wt % and in particular at least about 95 wt %, and preferably also
the weight ratio of water to ethanol is from about 5:1 to about 1:5, preferably from about 2:1 to about 1:2 and in particular from about 5:4 to about 4:5.

Another preferred subject matter of the present disclosure is therefore a cosmetic product comprising

a) a cosmetic preparation containing, in relation to its total weight,
a1) from about 60 to about 98 wt % of at least one polar solvent, wherein
the proportion by weight of water and ethanol in the total weight of the polar solvent a1) is preferably at least about 60 wt %, more preferably at least about 80 wt %, particularly preferably at least about 90 wt % and in particular at least about 95 wt %, and
the weight ratio of water to ethanol is from about 5:1 to about 1:5, preferably from about 2:1 to about 1:2 and in particular from about 5:4 to about 4:5,
   a2) from about 0.1 to about 20 wt % of at least one copolymer from the monomers
   i) N-tert-butylacrylamide
   ii) acrylic acid
   iii) ethyl acrylate
   iv) and optionally other monomers
   b) a device for flash evaporation of the cosmetic preparation a), comprising
   b1) a container b1) which can be closed and opened by employing a valve,
   b2) a heating device which makes it possible to heat a cosmetic preparation located in the container b1),
   b3) a nozzle b3) which enables atomization of the cosmetic preparation a).

A second essential constituent of cosmetic compositions as contemplated herein is the copolymer a2). With regard to the ease of preparation, ease of application and cosmetic effect of cosmetic compositions as contemplated herein, it has proven to be advantageous if the proportion by weight of the copolymer a2) in the total weight of the cosmetic preparation a) is from about 0.5 to about 15 wt %, preferably from about 1.0 to about 10 wt %.

The copolymer a2) can be ascribed to the monomers i) N-tert-butylacrylamide, ii) acrylic acid, iii) ethyl acrylate, and optionally other monomers.

Preferred copolymers a2) preferably consist to a proportion of at least about 90 wt %, preferably at least about 95 wt % and in particular at least about 97 wt % of the monomers i) N-tert-butylacrylamide, ii) acrylic acid, iii) ethyl acrylate. Particularly preferred copolymers a2) have been obtained exclusively from the monomers i) N-tert-butylacrylamide, ii) acrylic acid, iii) ethyl acrylate.

The above-described copolymers a2) are marketed for example under the name Ultrahold® 8 (INCI name: Acrylates/t-Butylacrylamide Copolymer; CAS number 26062-56-6) by BASF.

One particularly preferred subject matter of the present patent application is therefore a cosmetic product comprising

a) a cosmetic preparation containing, in relation to its total weight,
a1) from about 80 to about 98 wt % of at least one polar solvent;
a2) from about 0.1 to about 10 wt % of at least one copolymer from the monomers
i) N-tert-butylacrylamide
ii) acrylic acid
iii) ethyl acrylate;
b) a device for flash evaporation of the cosmetic preparation a).

The copolymer a2) is used in the cosmetic agents preferably in partially neutralized or neutralized form. Preferably at least one alkanolamine is used for the neutralization. The alkanolamines which can be used as the alkalinizing agent as contemplated herein are preferably selected from primary amines with a C₂-C₆-alkyl main body which carries at least one hydroxyl group. Particularly preferred alkanolamines are selected from the group formed of 2-aminoethan-1-ol (monoethanolamine), tris(2-hydroxyethyl)amine (triethanolamine), 3-aminopropan-1-ol, 4-aminobutan-1-ol, 5-aminopentan-1-ol, 1-aminopropan-2-ol, 1-aminobutan-2-ol, 1-aminopentan-2-ol, 1-aminopentan-3-ol, 1-aminopentan-4-ol, 3-amino-2-methylpropan-1-ol, 1-amino-2-methylpropan-2-ol, 3-aminopropane-1,2-diol, 2-amino-2-methylpropane-1,3-diol. Alkanolamines which are very particularly preferred as contemplated herein are selected from the group consisting of 2-aminoethan-1-ol, 2-amino-2-methylpropan-1-ol and 2-amino-2-methylpropane-1,3-diol. 2-Amino-2-methylpropanol has proven to be a particularly suitable neutralizing agent. Cosmetic agents which are preferred as contemplated herein contain at least one alkanolamine, preferably 2-amino-2-methylpropanol. 2-Amino-2-methylpropanol is used in the agents as contemplated herein preferably in an amount which does not exceed the amount required for the neutralization of the copolymer a2). The amount of 2-amino-2-methylpropanol used in the agents as contemplated herein is preferably from about 80 to about 100%, particularly preferably from about 90 to about 100% and in particular from about 95 to about 100% of the amount required for the complete neutralization of the copolymer a2). In one preferred embodiment, the proportion by weight of the 2-amino-2-methylpropanol in the total weight of the cosmetic preparation a) is from about 0.1 to about 2.0 wt %, preferably from about 0.1 to about 1.2 wt % and in particular from about 0.2 to about 0.8 wt %.

Besides the cosmetic preparation a), the cosmetic products as contemplated herein further comprise a device for flash evaporation. In the context of the present application, the expression “flash evaporation” refers to the creation of vapor as the pressure is lowered in a closed chamber filled with liquid, said chamber being at an overpressure (relative to the surrounding environment). Such an overpressure can be generated for example by heating a quantity of the cosmetic preparation a) in a closed chamber to a temperature T₁. At a given temperature T₁, the liquid in the closed chamber has a saturation pressure p₁. If the closed chamber is opened for example by employing a valve to a relaxation chamber which is not at an overpressure and which is at the pressure p₀<p₁, the pressure in the previously closed chamber decreases and the cosmetic preparation a), or the solvent or portions of said solvent contained in the cosmetic preparation, evaporates as the new pressure level spreads. The resulting vapor or spray mist can be used to apply specific cosmetic preparations.

Therefore, if the cosmetic preparation a) is heated in a closed chamber starting from standard conditions (T₀=25° C., p₀=1000 bar), this results in an increased pressure of the cosmetic preparation a) as well as an increased temperature. This increased pressure can be relieved in a relaxation chamber to a pressure p₀, for example the ambient air pressure (p₀=1000 bar), as a result of which an at least partial evaporation of the cosmetic preparation a) is achieved.

The cosmetic preparation a) may be relieved of pressure directly in the chamber in which it was previously heated. Alternatively, however, the heated cosmetic preparation a) at overpressure may also be transported, after heating, into a second chamber in which the pressure is then relieved.

In other words, flash evaporation is a method in which the cosmetic preparation a) is heated in a closed container by employing a heating device to temperatures above the ambient temperature, whereby a pressure above the ambient pressure is generated in the container, and the heated and pressurized cosmetic preparation a) is then released from the container into the environment.

A device for flash evaporation is accordingly a device which comprises a container and a heating device and which is designed in such a way that a cosmetic preparation a) in the closed container can be heated by employing the heating device to temperatures above the ambient temperature so that a pressure above the ambient pressure is generated in the container and the heated and pressurized cosmetic preparation a) can be released from the container into the environment.

At the same time as or after the pressure relief, the cosmetic preparation a) can be fed to a nozzle, such that for example properties of the vapor or spray mist produced by the flash evaporation can be influenced, in particular the droplet size or the droplet density but also the spray width and the shape of the spray cone. The use of nozzles, preferably atomizing nozzles, is therefore preferred. The specific nozzle type or the specific nozzle design is defined in a targeted manner as a function of the respective spray mist properties.

To sum up, a preferred device for flash evaporation has

b1) a container b1) which can be closed and opened by employing a valve and which defines the closed interior in which the cosmetic preparation can be accommodated,
b2) a heating device b2) which makes it possible to heat a cosmetic preparation located in the container b1).

Particular preference is given to the use of an additional nozzle b3) which enables an atomization of the cosmetic preparation a) escaping from the container. As an alternative to a valve, use can also be made of a closing element of comparable effect which is able to close or expose an associated opening in the container by employing a corresponding change in position.

One preferred subject matter of the present disclosure is a cosmetic product comprising

a) a cosmetic preparation containing, in relation to its total weight,
a1) from about 60 to about 98 wt % of at least one polar solvent;
a2) from about 0.1 to about 20 wt % of at least one copolymer from the monomers
i) N-tert-butylacrylamide
ii) acrylic acid
iii) ethyl acrylate
iv) and optionally other monomers;
b) a device for flash evaporation of the cosmetic preparation a), wherein the device for flash evaporation comprises a container b1) and a heating device b2) and is designed in such a way that
the cosmetic preparation a) can be accommodated in the interior of the container b1),
the interior of the container b1) which is at least partially filled with the cosmetic preparation a) can be closed,
the cosmetic preparation a) in the closed interior of the container b1) can be heated by employing the heating device b2), the pressure thereby being increased.

One particularly preferred subject matter of the present disclosure is therefore a cosmetic product comprising

a) a cosmetic preparation containing, in relation to its total weight,
a1) from about 60 to about 98 wt % of at least one polar solvent;
a2) from about 0.1 to about 20 wt % of at least one copolymer from the monomers
i) N-tert-butylacrylamide
ii) acrylic acid
iii) ethyl acrylate
iv) and optionally other monomers;
b) a device for flash evaporation of the cosmetic preparation a), comprising
b1) a container b1) which can be closed and opened by employing a valve,
b2) a heating device which makes it possible to heat a cosmetic preparation located in the container b1),
b3) a nozzle b3) which enables atomization of the cosmetic preparation a).

In other words, one particularly preferred subject matter of the present disclosure is a cosmetic product comprising

a) a cosmetic preparation containing, in relation to its total weight,
a1) from about 60 to about 98 wt % of at least one polar solvent;
a2) from about 0.1 to about 20 wt % of at least one copolymer from the monomers
i) N-tert-butylacrylamide
ii) acrylic acid
iii) ethyl acrylate
iv) and optionally other monomers;
b) a device for flash evaporation of the cosmetic preparation a), wherein the device for flash evaporation comprises a container b1) and a heating device b2) and is designed in such a way that
the cosmetic preparation a) can be accommodated in the interior of the container b1),
the interior of the container b1) which is at least partially filled with the cosmetic preparation a) can be closed,
the cosmetic preparation a) in the closed interior of the container b1) can be heated by employing the heating device b2), the pressure thereby being increased,
the heated cosmetic preparation a) can be released from the interior of the container b1) into the environment, the pressure thereby being reduced.

The container b1) in which the cosmetic preparation is heated is designed in such a way as to make it possible to close said container fully with respect to the surrounding environment during the heating of the cosmetic preparation a) and to open it after the heating in order to enable the flash evaporation of the cosmetic preparation a). This can be ensured for example by a flow control component, in particular a valve.

The container b1) in which the cosmetic preparation is heated is preferably in contact with a further container, from which the quantity of the cosmetic preparation intended for the flash evaporation is transferred into the container b1) prior to heating. The access between this storage container and the container b1) can be opened and closed by way of a suitable device, for example a valve. This further container is preferably designed in the form of a storage container, that is to say it preferably contains a multiple of, for example more than ten times, preferably more than fifty times, the quantity of the cosmetic preparation necessary for an evaporation operation. In other words, the further container/storage container preferably has a multiple, for example more than ten times the volume, preferably more than twenty times and in particular more than fifty times the volume of the container b1).

Another particularly preferred subject matter of the present disclosure is therefore a cosmetic product comprising

a) a cosmetic preparation containing, in relation to its total weight,
a1) from about 60 to about 98 wt % of at least one polar solvent;
a2) from about 0.1 to about 20 wt % of at least one copolymer from the monomers
i) N-tert-butylacrylamide
ii) acrylic acid
iii) ethyl acrylate
iv) and optionally other monomers;
b) a device for flash evaporation of the cosmetic preparation a), comprising
b1) a container b1) which can be closed and opened by employing a valve,
b2) a heating device which makes it possible to heat a cosmetic preparation located in the closed container b1),
b3) a nozzle b3) which enables atomization of the cosmetic preparation a),
c) a storage container for the cosmetic preparation a), from which the cosmetic preparation a) can pass into the container b1), wherein
the access between the storage container and the container b1) has a flow control component, such that the flow of the cosmetic preparation a) from the storage container into the container b1) can be interrupted;
the storage container has at least about ten times the volume, preferably at least about twenty times and in particular at least about fifty times the volume of the container b1).

The storage container is not a pressure container, and the cosmetic composition located in the storage container is not pressurized; in other words, the pressure in the interior of the storage container corresponds to the ambient pressure (also air pressure or atmospheric pressure). Such cosmetic products thus comprise no propellants for example. In addition, the cosmetic product does not have a pump device suitable for releasing or spraying the cosmetic preparation into the environment without the action of the device for flash evaporation.

One very particularly preferred subject matter of the present disclosure is therefore a cosmetic product comprising

a) a cosmetic preparation containing, in relation to its total weight,
a1) from about 60 to about 98 wt % of at least one polar solvent;
a2) from about 0.1 to about 20 wt % of at least one copolymer from the monomers
i) N-tert-butylacrylamide
ii) acrylic acid
iii) ethyl acrylate
iv) and optionally other monomers;
b) a device for flash evaporation of the cosmetic preparation a), comprising
b1) a container b1) which can be closed and opened by employing a valve,
b2) a heating device which makes it possible to heat a cosmetic preparation located in the closed container b1),
b3) a nozzle b3) which enables atomization of the cosmetic preparation a),
c) a storage container for the cosmetic preparation a), from which the cosmetic preparation a) can pass into the container b1), wherein
the access between the storage container and the container b1) has a flow control component, such that the flow of the cosmetic preparation a) from the storage container into the container b1) can be interrupted;
the storage container has at least about ten times the volume, preferably at least about fifty times the volume of the container b1);
the pressure in the interior of the storage container corresponds to the ambient pressure.

One very particularly preferred subject matter of the present disclosure is therefore a cosmetic product comprising

a) a cosmetic preparation containing, in relation to its total weight,
a1) from about 60 to about 98 wt % of at least one polar solvent;
a2) from about 0.1 to about 20 wt % of at least one copolymer from the monomers
i) N-tert-butylacrylamide
ii) acrylic acid
iii) ethyl acrylate
iv) and optionally other monomers;
b) a device for flash evaporation of the cosmetic preparation a), comprising
b1) a container b1) which can be closed and opened by employing a valve,
b2) a heating device which makes it possible to heat a cosmetic preparation located in the closed container b1),
b3) a nozzle b3) which enables atomization of the cosmetic preparation a),
c) a storage container for the cosmetic preparation a), from which the cosmetic preparation a) can pass into the container b1), wherein
the access between the storage container and the container b1) has a flow control component, such that the flow of the cosmetic preparation a) from the storage container into the container b1) can be interrupted;
the storage container has at least about ten times the volume, preferably at least about fifty times the volume of the container b1);
the pressure in the interior of the storage container corresponds to the ambient pressure and the cosmetic product does not include a propellant.

Preference is also given to cosmetic products comprising

a) a cosmetic preparation containing, in relation to its total weight,
a1) from about 60 to about 98 wt % of at least one polar solvent;
a2) from about 0.1 to about 20 wt % of at least one copolymer from the monomers
i) N-tert-butylacrylamide
ii) acrylic acid
iii) ethyl acrylate
iv) and optionally other monomers;
b) a device for flash evaporation of the cosmetic preparation a), comprising
b1) a container b1) which can be closed and opened by employing a valve,
b2) a heating device which makes it possible to heat a cosmetic preparation located in the closed container b1),
b3) a nozzle b3) which enables atomization of the cosmetic preparation a),
c) a storage container for the cosmetic preparation a), from which the cosmetic preparation a) can pass into the container b1), wherein
the access between the storage container and the container b1) has a flow control component, such that the flow of the cosmetic preparation a) from the storage container into the container b1) can be interrupted;
the storage container has at least about ten times the volume, preferably at least about fifty times the volume of the container b1);
the pressure in the interior of the storage container corresponds to the ambient pressure, wherein the cosmetic product does not include a pump device suitable for releasing or spraying the cosmetic preparation a) without the action of the device for flash evaporation.

To sum up, one particularly preferred subject matter of the present disclosure is therefore a cosmetic product comprising

a) a cosmetic preparation containing, in relation to its total weight,
a1) from about 60 to about 98 wt % of at least one polar solvent;
a2) from about 0.1 to about 20 wt % of at least one copolymer from the monomers
i) N-tert-butylacrylamide
ii) acrylic acid
iii) ethyl acrylate
iv) and optionally other monomers;
b) a device for flash evaporation of the cosmetic preparation a), comprising
b1) a container b1) which can be closed and opened by employing a valve,
b2) a heating device which makes it possible to heat a cosmetic preparation located in the closed container b1),
b3) a nozzle b3) which enables atomization of the cosmetic preparation a),
c) a storage container for the cosmetic preparation a), from which the cosmetic preparation a) can pass into the container b1), wherein
the access between the storage container and the container b1) has a flow control component, such that the flow of the cosmetic preparation a) from the storage container into the container b1) can be interrupted;
the storage container has at least about ten times the volume, preferably at least about fifty times the volume of the container b1);
the pressure in the interior of the storage container corresponds to the ambient pressure and the cosmetic product does not include a propellant,
   wherein the cosmetic product does not include a pump device suitable for releasing or spraying the cosmetic preparation a) without the action of the device for flash evaporation.

Besides the two constituents a1) and a2) described above, the cosmetic preparations a) as contemplated herein may contain further active ingredients or auxiliaries, particular preference being given to those active ingredients or auxiliaries which improve the ease of preparation, the ease of application and/or the cosmetic effect of cosmetic preparations as contemplated herein.

The poly(C₂-C₃)alkylene glycol-modified silicones a3) form a first group of preferred further active ingredients. Particular preference is given to poly(C₂-C₃)alkylene glycol-modified silicones from the group of alkoxylated dimethicones, in particular
ethoxylated dimethicones having the INCI name PEG-x Dimethicone, where x=2 to 20, preferably 3 to 17 and in particular 11 or 12;
ethoxylated dimethicones having the INCI name Bis-PEG-y Dimethicone, where x=3 to 25, preferably 4 to 20;
ethoxylated/propoxylated dimethicones having the INCI name PEG/PPG a/b Dimethicone, where a and b independently of one another represent numbers from 2 to 30, preferably from 12 to 24 and in particular from 14 to 20;
ethoxylated/propoxylated dimethicones having the INCI name Bis-PEG/PPG-c/d Dimethicone, where c and d independently of one another represent numbers from 10 to 25, preferably from 14 to 20 and in particular from 14 to 16;
ethoxylated/propoxylated dimethicones having the INCI name Bis-PEG/PPG-e/f PEG/PPG g/h Dimethicone, where e, f, g and h independently of one another represent numbers from 10 to 20, preferably 14 to 18 and in particular 16.

In a first preferred embodiment, the cosmetic preparation a) contains, in relation to its total weight, from about 0.01 to about 1.0 wt %, preferably from about 0.05 to about 0.8 wt % and in particular from about 0.1 to about 0.5 wt % of at least one poly(C₂-C₃)alkylene glycol-modified silicone a3).

Particular preference is given to the use of poly(C₂-C₃)alkylene glycol-modified silicone a3) from the group of alkoxylated dimethicones, preferably from the group of ethoxylated dimethicones and in particular from the group of compounds having the INCI name PEG-12 Dimethicone.

A further example of a preferred further active ingredient and auxiliary are the film-forming polymers a4), the use of which is particularly preferred in the cosmetic agents as contemplated herein.

Suitable film-forming polymers a4) are permanently and also temporarily cationic, anionic, nonionic or amphoteric polymers. These film-forming polymers may be of synthetic or natural origin. Preferred cosmetic preparations a) contain, in relation to their total weight, from about 0.1 to about 20 wt %, preferably from about 0.5 to about 15 wt % and in particular from about 1.0 to about 10 wt % of a film-forming polymer a4). This polymer a4) is different from the polymer a2).

Examples of customary film-forming polymers a4) are Acrylamide/Ammonium Acrylate Copolymer, Acrylamides/DMAPA Acrylates/Methoxy PEG Methacrylate Copolymer, Acrylamidopropyltrimonium Chloride/Acrylamide Copolymer, Acrylamidopropyltrimonium Chloride/Acrylates Copolymer, Acrylates/Acetoacetoxyethyl Methacrylate Copolymer, Acrylates/Acrylamide Copolymer, Acrylates/Ammonium Methacrylate Copolymer, Acrylates/t-Butylacrylamide Copolymer, Acrylates Copolymer, Acrylates/C1-2 Succinates/Hydroxyacrylates Copolymer, Acrylates/Lauryl Acrylate/Stearyl Acrylate/Ethylamine Oxide Methacrylate Copolymer, Acrylates/Octylacrylamide Copolymer, Acrylates/Octylacrylamide/Diphenyl Amodimethicone Copolymer, Acrylates/Stearyl Acrylate/Ethylamine Oxide Methacrylate Copolymer, Acrylates/VA Copolymer, Acrylates/VP Copolymer, Adipic Acid/Diethylenetriamine Copolymer, Adipic Acid/Dimethylaminohydroxypropyl Diethylenetriamine Copolymer, Adipic Acid/Epoxypropyl Diethylenetriamine Copolymer, Adipic Acid/Isophthalic Acid/Neopentyl Glycol/Trimethylolpropane Copolymer, Allyl Stearate/VA Copolymer, Aminoethylacrylate Phosphate/Acrylates Copolymer, Aminoethylpropanediol-Acrylates/Acrylamide Copolymer, Aminoethylpropanediol-AMPD-Acrylates/Diacetoneacrylamide Copolymer, Ammonium VA/Acrylates Copolymer, AMPD-Acrylates/Diacetoneacrylamide Copolymer, AMP-Acrylates/Allyl Methacrylate Copolymer, AMP-Acrylates/C1-18 Alkyl Acrylates/C1-8 Alkyl Acrylamide Copolymer, AMP-Acrylates/Diacetoneacrylamide Copolymer, AMP-Acrylates/Dimethylaminoethylmethacrylate Copolymer, Bacillus/Rice Bran Extract/Soybean Extract Ferment Filtrate, Bis-Butyloxyamodimethicone/PEG-60 Copolymer, Butyl Acrylate/Ethylhexyl Methacrylate Copolymer, Butyl Acrylate/Hydroxypropyl Dimethicone Acrylate Copolymer, Butylated PVP, Butyl Ester of Ethylene/MA Copolymer, Butyl Ester of PVM/MA Copolymer, Calcium/Sodium PVM/MA Copolymer, Corn Starch/Acrylamide/Sodium Acrylate Copolymer, Diethylene Glycolamine/Epichlorohydrin/Piperazine Copolymer, Dimethicone Crosspolymer, Diphenyl Amodimethicone, Ethyl Ester of PVM/MA Copolymer, Hydrolyzed Wheat Protein/PVP Crosspolymer, Isobutylene/Ethylmaleimide/Hydroxyethylmaleimide Copolymer, Isobutylene/MA Copolymer, Isobutylmethacrylate/Bis-Hydroxypropyl Dimethicone Acrylate Copolymer, Isopropyl Ester of PVM/MA Copolymer, Lauryl Acrylate Crosspolymer, Lauryl Methacrylate/Glycol Dimethacrylate Crosspolymer, MEA-Sulfite, Methacrylic Acid/Sodium Acrylamidomethyl Propane Sulfonate Copolymer, Methacryloyl Ethyl Betaine/Acrylates Copolymer, Octylacrylamide/Acrylates/Butylaminoethyl Methacrylate Copolymer, PEG/PPG-25/25 Dimethicone/Acrylates Copolymer, PEG-8/SMDI Copolymer, Polyacrylamide, Polyacrylate-6, Polybeta-Alanine/Glutaric Acid Crosspolymer, Polybutylene Terephthalate, Polyester-1, Polyethylacrylate, Polyethylene Terephthalate, Polymethacryloyl Ethyl Betaine, Polypentaerythrityl Terephthalate, Polyperfluoroperhydrophenanthrene, Polyquaternium-1, Polyquaternium-2, Polyquaternium-4, Polyquaternium-5, Polyquaternium-6, Polyquaternium-7, Polyquaternium-8, Polyquaternium-9, Polyquaternium-10, Polyquaternium-11, Polyquaternium-12, Polyquaternium-13, Polyquaternium-14, Polyquaternium-15, Polyquaternium-16, Polyquaternium-17, Polyquaternium-18, Polyquaternium-19, Polyquaternium-20, Polyquaternium-22, Polyquaternium-24, Polyquaternium-27, Polyquaternium-28, Polyquaternium-29, Polyquaternium-30, Polyquaternium-31, Polyquaternium-32, Polyquaternium-33, Polyquaternium-34, Polyquaternium-35, Polyquaternium-36, Polyquaternium-39, Polyquaternium-45, Polyquaternium-46, Polyquaternium-47, Polyquaternium-48, Polyquaternium-49, Polyquaternium-50, Polyquaternium-55, Polyquaternium-56, Polysilicone-9, Polyurethane-1, Polyurethane-6, Polyurethane-10, Polyvinyl Acetate, Polyvinyl Butyral, Polyvinylcaprolactam, Polyvinylformamide, Polyvinyl Imidazolinium Acetate, Polyvinyl Methyl Ether, Potassium Butyl Ester of PVM/MA Copolymer, Potassium Ethyl Ester of PVM/MA Copolymer, PPG-70 Polyglyceryl-10 Ether, PPG-12/SMDI Copolymer, PPG-51/SMDI Copolymer, PPG-10 Sorbitol, PVM/MA Copolymer, PVP, PVPNA/ltaconic Acid Copolymer, PVP/VA/Vinyl Propionate Copolymer, Rhizobian Gum, Rosin Acrylate, Shellac, Sodium Butyl Ester of PVM/MA Copolymer, Sodium Ethyl Ester of PVM/MA Copolymer, Sodium Polyacrylate, Sterculia Urens Gum, Terephthalic Acid/Isophthalic Acid/Sodium Isophthalic Acid Sulfonate/Glycol Copolymer, Trimethylolpropane Triacrylate, Trimethylsiloxysilylcarbamoyl Pullulan, VA/Crotonates Copolymer, VA/Crotonates/Methacryloxybenzophenone-1 Copolymer, VA/Crotonates/Vinyl Neodecanoate Copolymer, VA/Crotonates/Vinyl Propionate Copolymer, VA/DBM Copolymer, VA/Vinyl Butyl Benzoate/Crotonates Copolymer, Vinylamine/Vinyl Alcohol Copolymer, Vinyl Caprolactam/VP/Dimethylaminoethyl Methacrylate Copolymer, VP/Acrylates/Lauryl Methacrylate Copolymer, VP/Dimethylaminoethylmethacrylate Copolymer, VP/DMAPA Acrylates Copolymer, VP/Hexadecene Copolymer, VP/VA Copolymer, VP/Vinyl Caprolactam/DMAPA Acrylates Copolymer, Yeast Palmitate and Styrene/VP Copolymer.

Particular preference is given to cosmetic products which are characterized in that the film-forming polymer a4) is selected from the group of anionic polymers, preferably from the group of copolymers of acrylic acid and methacrylic acid.

One particularly preferred anionic acrylate copolymer a4) is composed at least of the following monomer units: at least one (meth)acrylic acid unit (1), at least one (meth)acrylic acid alkyl ester unit (2) and at least one (meth)acrylic acid hydroxyalkyl ester unit (3). As contemplated herein, this preferred copolymer a4) may be composed of further monomer units. According to embodiments of the invention, however, the copolymer a4) is composed only of units (1), (2) and (3), that is to say it consists of units derived from these monomer units.

The at least one methacrylic acid unit (1) may be a methacrylic acid or acrylic acid unit.

The alkyl residue of the (meth)acrylic acid alkyl ester unit (2) is preferably a C1-C8 alkyl residue, which may be linear or branched. Examples of alkyl residues are methyl, ethyl, propyl, isopropyl, 1-butyl, 2-butyl, isobutyl, tert-butyl, linear or branched pentyl, linear or branched hexyl, linear or branched heptyl, and linear or branched octyl. More preferably, the alkyl group is a C1 to C5 alkyl group. According to one embodiment of the invention, two or more (meth)acrylic acid alkyl ester units (2) are contained, which differ in terms of the carbon number of the alkyl group. For example, a C1-C3 alkyl methacrylate unit and a C2-C5 alkyl acrylate unit are contained.

The hydroxyalkyl residue of the (meth)acrylic acid hydroxyalkyl ester unit (3) may be a hydroxy-C1-C10-alkyl residue, preferably a hydroxy-C2-C5-alkyl residue. In one preferred embodiment, the (meth)acrylic acid hydroxyalkyl ester unit (3) is (meth)acrylic acid hydroxyethyl ester.

The proportion of the units (1), (2) and (3) in the acrylate resin a4) can vary within wide limits. The proportion of the unit (1) in the acrylate copolymer is preferably from about 2 to 5 about 0 wt %, more preferably from about 5 to about 30 wt %. The proportion of the unit (2) in the acrylate copolymer is preferably from about 5 to about 95 wt %, more preferably from about 45 to about 90 wt %. The proportion of the unit (3) in the acrylate copolymer is preferably from about 2 to about 70 wt %, more preferably from about 5 to about 30 wt %.

Suitable anionic acrylate copolymers a4) are commercially available under the INCI name Acrylates/Hydroxyesters Acrylates Copolymer. Most preferably, the anionic acrylate copolymer (a) is Acudyne® 1000 from The Dow Chemical Company.

Another preferred anionic acrylate copolymer a4) comprises structural units of formula (4) in which R¹ is a methyl group and R² is a methyl group, and structural units of formula (4) in which R¹ is a hydrogen atom and R² is a butyl group (in particular an n-butyl group), and structural units of formula (5) in which R³ is a methyl group and R⁴ is a 2-hydroxyethyl group, and structural units of formula (6) in which R⁷ is a methyl group, at least one structural unit of formula (7) in which R⁵ and R⁶ independently of one another are a hydrogen atom or a (C₁ to C₆) alkyl group, preferably hydrogen

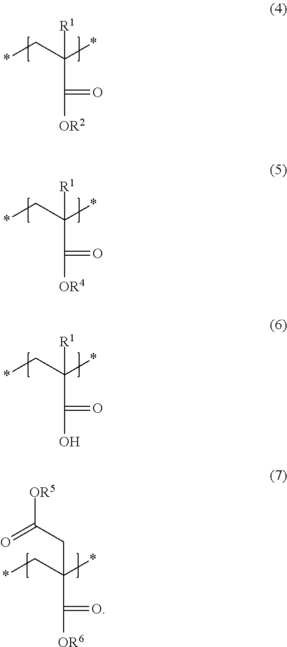

One particularly preferred polymer has the INCI nomenclature Acrylates/C1-2 Succinates/Hydroxyacrylates Copolymer. It can be purchased for example from the company Dow under the trade name Acudyne LT-120 (INCI nomenclature: Acrylates/C1-2 Succinates/Hydroxyacrylates Copolymer).

A second group of polymers a4) which are used with preference in the cosmetic preparation are the vinylpyrrolidone homopolymers or copolymers B. Polymers B which are used with particular preference are for example:
polyvinylpyrrolidones, such as those marketed for example under the name Luviskol® (BASF),
vinylpyrrolidone/vinyl ester copolymers, such as those marketed for example under the trademark Luviskol® (BASF). Luviskol® VA 64 and Luviskol® VA 73, which are in each case vinylpyrrolidone/vinyl acetate copolymers, are preferred nonionic polymers.

Due to their cosmetic effect in combination with the copolymers a2), film-forming polymers which are preferably used as contemplated herein are, in particular, the polyvinylpyrrolidones (INCI name: PVP) and the vinylpyrrolidone/vinyl acetate copolymers (INCI name VP/VA Copolymer). Preferred cosmetic products are characterized in that the film-forming polymer a4) is selected from the group of nonionic polymers, preferably from the group of polyvinylpyrrolidones and vinylpyrrolidone/vinyl acetate copolymers, preferably polyvinylpyrrolidones.

A third group of particularly preferred film-forming polymers a4) comprises amphoteric fixative polymers C based on

at least one monomer C1 selected from acrylic acid, methacrylic acid, alkyl acrylates and alkyl methacrylates, and
at least one amphoteric monomer C2 of formula C2

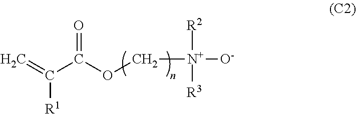

wherein
R¹ is H or CH₃,
R² and R³ independently of one another are each optionally branched C₁₋₁₀ alkyl and
n is an integer from 1 to 20.

In the context of the present embodiment of the invention, additional amphoteric fixative polymers which are formed from the aforementioned monomers will be understood to mean only those copolymers C which contain, besides polymer units resulting from the incorporation of the aforementioned monomers C1 and C2 into the copolymer, at most 5 wt %, preferably 1 wt %, polymer units which can be ascribed to the incorporation of other monomers. The copolymers C are preferably composed exclusively of polymer units resulting from the incorporation of the aforementioned monomers C1 and C2 into the copolymer.

Suitable monomers C1 are acrylic acid, methacrylic acid, C₁₋₂₀ alkyl acrylates and C₁₋₂₀ alkyl methacrylates. Monomer C1 is particularly suitably selected from acrylic acid, methacrylic acid, methyl acrylate, methyl methacrylate, ethyl acrylate, ethyl methacrylate, propyl acrylate, propyl methacrylate, isopropyl acrylate, isopropyl methacrylate, lauryl acrylate, lauryl methacrylate, cetyl acrylate, cetyl methacrylate, stearyl acrylate and stearyl methacrylate, very particularly preferably from acrylic acid, methacrylic acid, methyl acrylate, methyl methacrylate, ethyl acrylate, ethyl methacrylate, lauryl acrylate, lauryl methacrylate, stearyl acrylate and stearyl methacrylate.

Suitable monomers C2 are (meth)acryloylalkylamine oxides of formula C2 in which R² and R³ independently of one another are each methyl, ethyl, propyl, isopropyl, butyl, isobutyl or tert-butyl, particularly preferably methyl. Suitable monomers C2 are also selected from at least one monomer from the group formed of (meth)acryloylalkylamine oxides of formula C2 in which n is an integer from 1 to 5, preferably an integer from 1 to 3, and particularly preferably 2.

Preference is also given to monomers C2 selected from at least one monomer from the group formed of (meth)acryloylalkylamine oxides of formula C2 in which R¹ is CH₃.

The monomers C2 are particularly suitably selected from at least one monomer from the group formed of (meth)acryloylalkylamine oxides of formula C2 in which R² and R³ independently of one another are methyl, ethyl, propyl, isopropyl, isobutyl or tert-butyl, particularly preferably methyl, n is in each case an integer from 1 to 5, preferably an integer from 1 to 3 and particularly preferably 2, and R¹ is in each case CH₃. Monomer C2 is very particularly suitably selected from at least one monomer from the group formed of (meth)acryloylalkylamine oxides of formula C2 in which R′, R² and R³ are CH₃ and n is 2. Preferred cosmetic products are characterized in that the film-forming polymer a4) is selected from the group of the copolymers of methacryloylethyl-N,N-dimethylamine oxide.

In one particularly suitable embodiment, the cosmetic preparation as contemplated herein contains at least one amphoteric fixative polymer which is formed of
at least two monomers C1, wherein the first monomer is selected from acrylic acid, methacrylic acid, methyl acrylate, methyl methacrylate, ethyl acrylate, ethyl methacrylate, propyl acrylate, propyl methacrylate, isopropyl acrylate and isopropyl methacrylate, and the second monomer is selected from stearyl acrylate and stearyl methacrylate, and
as the monomer C2 methacryloylethylamine oxide, in particular methacryloylethyl-N,N-dimethylamine oxide (in formula (A2): R¹=CH₃, n=2, R² and R³=CH₃).

These copolymers are also known and are available for example under the name Diaformer Z-632 from the company Clariant, particular preference being given to the use of Diaformer Z-632.

In one suitable embodiment, the agent as contemplated herein contains at least one amphoteric fixative polymer which is formed of
at least three monomers C1, wherein the first monomer is selected from acrylic acid, methacrylic acid, methyl acrylate, methyl methacrylate, ethyl acrylate, ethyl methacrylate, propyl acrylate, propyl methacrylate, isopropyl acrylate and isopropyl methacrylate, the second monomer is selected from lauryl acrylate and lauryl methacrylate, and the third monomer is selected from stearyl acrylate and stearyl methacrylate, and as monomer C2 methacryloylethylamine oxide, in particular methacryloylethyl-N,N-dimethylamine oxide (in formula (A2): R¹=CH₃, n=2, R² and R³=CH₃).

Such copolymers are likewise known and are available for example under the names Diaformer Z-611, Diaformer Z-612, Diaformer Z-613, Diaformer Z-631, Diaformer Z-633, Diaformer Z-651, Diaformer Z-711N, Diaformer Z-712N and Diaformer Z-731N from the company Clariant, preference being given to the use of Diaformer Z-712N and Diaformer Z-651.

A fourth group of particularly preferred film-forming polymers a4) comprises copolymers a4) which can be ascribed to the monomers i) N-tert-octylacrylamide, ii) acrylic acid, iii) tert-butylaminoethyl methacrylate, and optionally other monomers.

Preferred copolymers a4) preferably consist to a proportion of at least about 90 wt %, preferably at least about 95 wt %, and in particular at least about 97 wt % of the monomers N-tert-octylacrylamide, acrylic acid and tert-butylaminoethyl methacrylate. Particularly preferred copolymers a4) were obtained exclusively from the monomers N-tert-octylacrylamide, acrylic acid and tert-butylaminoethyl methacrylate.

Particular preference is given to copolymers a4) composed of the monomers i) N-tert-octylacrylamide, ii) acrylic acid, iii) tert-butylaminoethyl methacrylate, iv) methyl methacrylate and v) hydroxypropyl methacrylate.

The copolymers a4) described above are marketed for example under the name Amphomer® (INCI name: Octylacrylamide/Acrylates/Butylaminoethyl Methacrylate Copolymer, CAS number 70801-07-9) by the company National Starch.

As further suitable active ingredients or auxiliaries, mention may be made in particular of additional care substances.

The agent may contain for example, as a care substance, at least one protein hydrolysate and/or one of the derivatives thereof. Protein hydrolysates are product mixtures which are obtained by acid-, base-, or enzyme-catalyzed breakdown of proteins. As contemplated herein, the term protein hydrolysates will also be understood to mean total hydrolysates and also individual amino acids and derivatives thereof, as well as mixtures of different amino acids. The molecular weight of the protein hydrolysates which can be used as contemplated herein is between about 75, the molecular weight for glycine, and about 200,000; the molecular weight is preferably from about 75 to about 50,000 and very particularly preferably from about 75 to about 20,000 daltons.

The agent as contemplated herein may also contain, as a care substance, at least one vitamin, one provitamin, one vitamin precursor and/or one of the derivatives thereof. As contemplated herein, preference is given to those vitamins, provitamins and vitamin precursors which are usually assigned to groups A, B, C, E, F and H.

Further care substances are panthenol, caffeine, nicotinamide and sorbitol.

The agents as contemplated herein may also contain, as a care substance, at least one plant extract, but also mono- or oligosaccharides and/or lipids.

The composition of some particularly preferred cosmetic preparations as contemplated herein can be seen in the following tables (unless indicated otherwise, figures specified in wt % are based on the total weight of the cosmetic agent). With regard to further preferred embodiments of these particularly preferred compositions, what has been stated above in relation to the cosmetic preparations a) as contemplated herein applies, mutatis mutandis.

| | Formula 1 | Formula 2 | Formula 3 | Formula 4 | Formula 5 |
|---|---|---|---|---|---|
| Polar solvent a1) | 60 to 98 | 70 to 98 | 80 to 98 | 81 | 95 |
| Copolymer a2) | 0.1 to 20 | 0.5 to 15 | 1.0 to 10 | 8.5 | 3.6 |
| Optional additives | ad 100 | ad 100 | ad 100 | ad 100 | ad 100 |
| | Formula 6 | Formula 7 | Formula 8 | Formula 9 | Formula 10 |
| Polar solvent a1) | 60 to 98 | 70 to 98 | 80 to 98 | 81 | 95 |
| Copolymer a2) ¹⁾ | 0.1 to 20 | 0.5 to 15 | 1.0 to 10 | 8.5 | 3.6 |
| Optional additives | ad 100 | ad 100 | ad 100 | ad 100 | ad 100 |
| | Formula 11 | Formula 12 | Formula 13 | Formula 14 | Formula 15 |
| Polar solvent a1) | 60 to 98 | 70 to 98 | 80 to 98 | 81 | 95 |
| Copolymer a2) | 0.1 to 20 | 0.5 to 15 | 1.0 to 10 | 8.5 | 3.6 |
| Poly(C₂-C₃)alkylene | 0.01 to 1.0 | 0.05 to 0.8 | 0.1 to 0.5 | 0.28 | 0.12 |
| glycol-modified silicone | | | | | |
| a3) | | | | | |
| Optional additives | ad 100 | ad 100 | ad 100 | ad 100 | ad 100 |
| | Formula 16 | Formula 17 | Formula 18 | Formula 19 | Formula 20 |
| Polar solvent a1) | 60 to 98 | 70 to 98 | 80 to 98 | 81 | 95 |
| Copolymer a2) ¹⁾ | 0.1 to 20 | 0.5 to 15 | 1.0 to 10 | 8.5 | 3.6 |
| PEG-12 Dimethicone | 0.01 to 1.0 | 0.05 to 0.8 | 0.1 to 0.5 | 0.28 | 0.12 |
| Optional additives | ad 100 | ad 100 | ad 100 | ad 100 | ad 100 |
| | Formula 21 | Formula 22 | Formula 23 | Formula 24 | Formula 25 |
| Polar solvent a1) | 60 to 98 | 70 to 98 | 80 to 98 | 81 | 95 |
| Copolymer a2) | 0.1 to 20 | 0.5 to 15 | 1.0 to 10 | 8.5 | 3.6 |
| Polymer a4) | 0.1 to 20 | 0.5 to 15 | 1.0 to 10 | 3.9 | 0.9 |
| Optional additives | ad 100 | ad 100 | ad 100 | ad 100 | ad 100 |
| | Formula 26 | Formula 27 | Formula 28 | Formula 29 | Formula 30 |
| Polar solvent a1) | 60 to 98 | 70 to 98 | 80 to 98 | 81 | 93 |
| Copolymer a2) ¹⁾ | 0.1 to 20 | 0.5 to 15 | 1.0 to 10 | 8.5 | 3.6 |
| (Meth)acrylic acid | 0.1 to 20 | 0.5 to 15 | 1.0 to 10 | 3.9 | 0.9 |
| copolymer | | | | | |
| Optional additives | ad 100 | ad 100 | ad 100 | ad 100 | ad 100 |
| | Formula 31 | Formula 32 | Formula 33 | Formula 34 | Formula 35 |
| Polar solvent a1) | 60 to 98 | 7 0 to 98 | 80 to 98 | 81 | 93 |
| Copolymer a2) | 0.1 to 20 | 0.5 to 15 | 1.0 to 10 | 8.5 | 3.6 |
| Vinylpyrrolidone/vinyl | 0.1 to 20 | 0.5 to 15 | 1.0 to 10 | 3.9 | 0.9 |
| acetate copolymer | | | | | |
| Optional additives | ad 100 | ad 100 | ad 100 | ad 100 | ad 100 |
| | Formula 36 | Formula 37 | Formula 38 | Formula 39 | Formula 40 |
| Polar solvent a1) | 60 to 98 | 70 to 98 | 80 to 98 | 81 | 93 |
| Copolymer a2) ¹⁾ | 0.1 to 20 | 0.5 to 15 | 1.0 to 10 | 8.5 | 3.6 |
| (Meth)acryloylethyl- | 0.1 to 20 | 0.5 to 15 | 1.0 to 10 | 3.9 | 0.9 |
| N,N-dimethylamine | | | | | |
| oxide copolymer | | | | | |
| Optional additives | ad 100 | ad 100 | ad 100 | ad 100 | ad 100 |
| | Formula 41 | Formula 42 | Formula 43 | Formula 44 | Formula 45 |
| Polar solvent a1) | 60 to 98 | 70 to 98 | 80 to 98 | 81 | 93 |
| Copolymer a2) | 0.1 to 20 | 0.5 to 15 | 1.0 to 10 | 8.5 | 3.6 |
| N-tert-octylacrylamide/- | 0.1 to 20 | 0.5 to 15 | 1.0 to 10 | 3.9 | 0.9 |
| acrylic acid/tert- | | | | | |
| butylaminoethyl | | | | | |
| methacrylate copolymer | | | | | |
| Optional additives | ad 100 | ad 100 | ad 100 | ad 100 | ad 100 |
| | Formula 46 | Formula 47 | Formula 48 | Formula 49 | Formula 50 |
| Polar solvent a1) | 60 to 98 | 70 to 98 | 80 to 97 | 81 | 93 |
| Copolymer a2) | 0.1 to 20 | 0.5 to 15 | 1.0 to 10 | 8.5 | 3.6 |
| Poly(C₂-C₃)alkylene | 0.01 to 1.0 | 0.05 to 0.8 | 0.1 to 0.5 | 0.28 | 0.12 |
| glycol-modified silicone | | | | | |
| a3) | | | | | |
| Polymer a4) | 0.1 to 20 | 0.5 to 15 | 1.0 to 10 | 3.9 | 0.9 |
| Optional additives | ad 100 | ad 100 | ad 100 | ad 100 | ad 100 |
| | Formula 51 | Formula 52 | Formula 53 | Formula 54 | Formula 55 |
| Polar solvent a1) | 60 to 98 | 70 to 98 | 80 to 97 | 81 | 93 |
| Copolymer a2) | 0.1 to 20 | 0.5 to 15 | 1.0 to 10 | 8.5 | 3.6 |
| PEG-12 Dimethicone | 0.01 to 1.0 | 0.05 to 0.8 | 0.1 to 0.5 | 0.28 | 0.12 |
| Polymer a4) | 0.1 to 20 | 0.5 to 15 | 1.0 to 10 | 3.9 | 0.9 |
| Optional additives | ad 100 | ad 100 | ad 100 | ad 100 | ad 100 |
| | Formula 56 | Formula 57 | Formula 58 | Formula 59 | Formula 60 |
| Polar solvent a1) | 60 to 98 | 70 to 98 | 80 to 97 | 81 | 93 |
| Copolymer a2) ¹⁾ | 0.1 to 20 | 0.5 to 15 | 1.0 to 10 | 8.5 | 3.6 |
| PEG-12 Dimethicone | 0.01 to 1.0 | 0.05 to 0.8 | 0.1 to 0.5 | 0.28 | 0.12 |
| Polymer a4) | 0.1 to 20 | 0.5 to 15 | 1.0 to 10 | 3.9 | 0.9 |
| Optional additives | ad 100 | ad 100 | ad 100 | ad 100 | ad 100 |
| | Formula 61 | Formula 62 | Formula 63 | Formula 64 | Formula 65 |
| Polar solvent a1) | 60 to 98 | 70 to 98 | 80 to 97 | 81 | 93 |
| Copolymer a2) | 0.1 to 20 | 0.5 to 15 | 1.0 to 10 | 8.5 | 3.6 |
| Poly(C₂-C₃)alkylene | 0.01 to 1.0 | 0.05 to 0.8 | 0.1 to 0.5 | 0.28 | 0.12 |
| glycol-modified silicone | | | | | |
| a3) | | | | | |
| (Meth)acrylic acid | 0.1 to 20 | 0.5 to 15 | 1.0 to 10 | 3.9 | 0.9 |
| copolymer | | | | | |
| Optional additives | ad 100 | ad 100 | ad 100 | ad 100 | ad 100 |
| | Formula 66 | Formula 67 | Formula 68 | Formula 69 | Formula 70 |
| Polar solvent a1) | 60 to 98 | 70 to 98 | 80 to 97 | 81 | 93 |
| Copolymer a2) | 0.1 to 20 | 0.5 to 15 | 1.0 to 10 | 8.5 | 3.6 |
| Poly(C₂-C₃)alkylene | 0.01 to 1.0 | 0.05 to 0.8 | 0.1 to 0.5 | 0.28 | 0.12 |
| glycol-modified silicone | | | | | |
| a3) | | | | | |
| Vinylpyrrolidone/vinyl | 0.1 to 20 | 0.5 to 15 | 1.0 to 10 | 3.9 | 0.9 |
| acetate copolymer | | | | | |
| Optional additives | ad 100 | ad 100 | ad 100 | ad 100 | ad 100 |
| | Formula 71 | Formula 72 | Formula 73 | Formula 74 | Formula 75 |
| Polar solvent a1) | 60 to 98 | 70 to 98 | 80 to 97 | 81 | 93 |
| Copolymer a2) | 0.1 to 20 | 0.5 to 15 | 1.0 to 10 | 8.5 | 3.6 |
| Poly(C₂-C₃)alkylene | 0.01 to 1.0 | 0.05 to 0.8 | 0.1 to 0.5 | 0.28 | 0.12 |
| glycol-modified silicone | | | | | |
| a3) | | | | | |
| (Meth)acrylic acid | 0.1 to 20 | 0.5 to 15 | 1.0 to 10 | 3.9 | 0.9 |
| copolymer | | | | | |
| Optional additives | ad 100 | ad 100 | ad 100 | ad 100 | ad 100 |
| | Formula 76 | Formula 77 | Formula 78 | Formula 79 | Formula 80 |
| Polar solvent a1) | 60 to 98 | 70 to 98 | 80 to 97 | 81 | 93 |
| Copolymer a2) | 0.1 to 20 | 0.5 to 15 | 1.0 to 10 | 8.5 | 3.6 |
| Poly(C₂-C₃)alkylene | 0.01 to 1.0 | 0.05 to 0.8 | 0.1 to 0.5 | 0.28 | 0.12 |
| glycol-modified silicone | | | | | |
| a3) | | | | | |
| (Meth)acryloylethyl- | 0.1 to 20 | 0.5 to 15 | 1.0 to 10 | 3.9 | 0.9 |
| N,N-dimethylamine | | | | | |
| oxide copolymer | | | | | |
| Optional additives | ad 100 | ad 100 | ad 100 | ad 100 | ad 100 |
| | Formula 81 | Formula 82 | Formula 83 | Formula 84 | Formula 85 |
| Polar solvent a1) | 60 to 98 | 70 to 98 | 80 to 97 | 81 | 93 |
| Copolymer a2) | 0.1 to 20 | 0.5 to 15 | 1.0 to 10 | 8.5 | 3.6 |
| Poly(C₂-C₃)alkylene | 0.01 to 1.0 | 0.05 to 0.8 | 0.1 to 0.5 | 0.28 | 0.12 |
| glycol-modified silicone | | | | | |
| a3) | | | | | |
| N-tert-octylacrylamide/- | 0.1 to 20 | 0.5 to 15 | 1.0 to 10 | 3.9 | 0.9 |
| acrylic acid/tert- | | | | | |
| butylaminoethyl | | | | | |
| methacrylate copolymer | | | | | |
| Optional additives | ad 100 | ad 100 | ad 100 | ad 100 | ad 100 |

¹⁾ Copolymer having the INCI name Acrylates/t-Butylacrylamide Copolymer

Very particularly preferred cosmetic preparations contain, besides the above-described constituents a1) to a4), only small amounts of further active ingredients and auxiliaries. On account of their ease of preparation and good cosmetic effect, particular preference is given to cosmetic preparations which are characterized in that the proportion by weight of the constituents a1), a2) and, if present, the optional constituents a3) and/or a4) in relation to the total weight of the cosmetic preparation is at least about 80 wt %, preferably at least about 90 wt %, particularly preferably at least about 93 wt % and in particular at least about 97 wt %. Very particularly preferred cosmetic preparations consist to a proportion of at least about 80 wt %, preferably at least about 90 wt % and in particular at least about 97 wt % of the constituents a1), a2) and a3), in relation to the total weight of the preparation.

As stated in the introduction, the cosmetic preparations a) as contemplated herein are particularly suitable for application by employing a device for flash evaporation. A further subject matter of the present application is therefore the use of a cosmetic preparation a) containing, in relation to its total weight,

a1) from about 60 to about 98 wt % of at least one polar solvent;
a2) from about 0.1 to about 20 wt % of at least one copolymer from the monomers
i) N-tert-butylacrylamide
ii) acrylic acid
iii) ethyl acrylate
iv) and optionally other monomers
as process material in a device for flash evaporation.

Another subject matter of the present disclosure is the use of a product as contemplated herein for applying a cosmetic preparation a) to keratin-containing fibers, in particular human hair, or for the temporary shaping of keratin-containing fibers, in particular human hair.

A method for the temporary shaping of keratin-containing fibers, in particular human hair, in which a cosmetic preparation a) containing, in relation to its total weight,

a1) from about 60 to about 98 wt % of at least one polar solvent;
a2) from about 0.1 to about 20 wt % of at least one copolymer from the monomers
i) N-tert-butylacrylamide
ii) acrylic acid
iii) ethyl acrylate
iv) and optionally other monomers
is applied to the keratin-containing fibers by employing a device for flash evaporation, forms a further subject matter of the present application. By employing the device for flash evaporation, the cosmetic preparation a) is preferably converted into a spray mist which is then applied to the keratin-containing fibers.

In order to achieve a sufficient spraying effect, the cosmetic preparation a) is preferably heated to temperatures above the boiling point of the polar solvent or solvent mixture contained in the cosmetic preparation a).

If the polar solvent is water or if the solvent mixture has a water content above about 50 wt % (in relation to the total weight of the solvent mixture), the cosmetic preparation is preferably heated to temperatures above about 100° C., more preferably to temperatures of from about 100° C. to about 240° C., particularly preferably to temperatures of from about 140° C. to about 160° C.

In cases where the polar solvent is water or a solvent mixture having a water content above 50 wt % (in relation to the total weight of the solvent mixture), the overpressure achieved as a result of heating the cosmetic preparation a) is preferably between about 1.1 and about 8 bar, more preferably between about 1.2 and about 4 bar.

One preferred subject matter of the application is a method for the temporary shaping of keratin-containing fibers, in particular human hair, in which a cosmetic preparation a) containing, in relation to its total weight,

a1) from about 60 to about 98 wt % of at least one polar solvent;
a2) from about 0.1 to about 20 wt % of at least one copolymer from the monomers
i) N-tert-butylacrylamide
ii) acrylic acid
iii) ethyl acrylate
iv) and optionally other monomers
is applied to the keratin-containing fibers by employing a device for flash evaporation, wherein,
from a storage container in the interior of which a pressure corresponding to the ambient pressure prevails, a partial quantity of the cosmetic preparation a) located in this storage container is transferred into a container b1);
then the access between the storage container and the container b1) is interrupted by a flow control component, such that the flow of the cosmetic preparation a) from the storage container into the container b1) can be interrupted;
then the cosmetic preparation a) located in the container b1) which is sealed off from the environment is heated by employing a heating device so that the pressure in the interior of the container b1) increases to values above the ambient pressure, preferably to values between about 1.1 and about 8 bar, in particular to values between about 1.2 and about 4 bar;
then the container b1) which is at a pressure above the ambient pressure is opened in such a way as to enable at least a partial quantity, preferably at least about 50 wt %, more preferably at least about 80 wt % and in particular at least about 90 wt %, of the cosmetic preparation located in the container b1) to be released from the container b1) into the environment, thereby reducing the pressure prevailing in the container b1) at the time of opening of the container.

The release of the cosmetic preparation a) into the environment preferably takes place by forming a spray mist of the cosmetic preparation a).

The cosmetic preparation a) released from the container b1) is preferably applied to keratin-containing fibers, in particular human hair.

Particular preference is given to methods during which the cosmetic preparation released from the container b1) is conducted through a nozzle before being applied to the keratin-containing fibers.

With regard to further preferred embodiments of the uses as contemplated herein and of the method as contemplated herein, what has been stated above in relation to the cosmetic preparations a) as contemplated herein and in relation to the device for flash evaporation b) applies, mutatis mutandis.

The agents, uses and methods as contemplated herein, and some preferred embodiments thereof, are characterized in summary by the following points:

1. A cosmetic product comprising
a) a cosmetic preparation containing, in relation to its total weight,
a1) from about 60 to about 98 wt % of at least one polar solvent;
a2) from about 0.1 to about 20 wt % of at least one copolymer from the monomers
i) N-tert-butylacrylamide
ii) acrylic acid
iii) ethyl acrylate
iv) and optionally other monomers;
b) a device for flash evaporation of the cosmetic preparation a).
2. The cosmetic product according to point 1, characterized in that the device for flash evaporation comprises a container b1) and a heating device b2) and is designed in such a way that
the cosmetic preparation a) can be accommodated in the interior of the container b1),
the interior of the container b1) at least partially filled with the cosmetic preparation a) can be closed,
the cosmetic preparation a) in the closed interior of the container b1) can be heated by employing the heating device b2), the pressure thereby being increased,
the heated cosmetic preparation a) can be released from the interior of the container b1) into the environment, the pressure thereby being reduced.
   3. The cosmetic product according to any of the preceding points, characterized in that the proportion by weight of the polar solvent a1) in relation to the total weight of the cosmetic preparation a) is from about 70 to about 98 wt %, preferably from about 80 to about 98 wt %.
   4. The cosmetic product according to any of the preceding points, characterized in that the polar solvent a1) has a boiling point (20° C., 1013 mbar) between about 50 and about 110° C., preferably between about 70 and about 105° C.
   5. The cosmetic product according to any of the preceding points, characterized in that the polar solvent a1) is selected from the group consisting of ethanol, isopropanol and water.
   6. The cosmetic product according to any of the preceding points, characterized in that the proportion by weight of water and ethanol in relation to the total weight of the polar solvent a1) is preferably at least about 60 wt %, more preferably at least about 80 wt %, particularly preferably at least about 90 wt % and in particular at least about 95 wt %.
   7. The cosmetic product according to any of the preceding points, characterized in that the proportion by weight of water in relation to the total weight of the polar solvent a1) is more than about 80 wt %, preferably more than about 88 wt % and in particular more than about 92 wt %.
   8. The cosmetic product according to any of the preceding points, characterized in that the polar solvent a1) consists of ethanol to a proportion of more than about 70 wt %, preferably more than about 80 wt % and in particular more than about 90 wt %, based on the weight of the polar solvent.
   9. The cosmetic product according to any of the preceding points, characterized in that the proportion by weight of ethanol in relation to the total weight of the cosmetic preparation is at most 55 wt %, preferably from about 10 to about 55 wt %, particularly preferably from about 25 to about 55 wt % and in particular from about 40 to about 55 wt %.
   10. The cosmetic product according to any of the preceding points, characterized in that the polar solvent a1) comprises water and ethanol and the weight ratio of water to ethanol is from about 5:1 to about 1:5, preferably from about 2:1 to about 1:2 and in particular from about 5:4 to about 4:5.
   11. The cosmetic product according to any of the preceding points, characterized in that the proportion by weight of the copolymer a2) in the total weight of the cosmetic preparation a) is from about 0.5 to about 15 wt %, preferably from about 1.0 to about 10 wt %.
   12. The cosmetic product according to any of the preceding points, characterized in that the cosmetic preparation a) contains, in relation to its total weight, from about 0.01 to about 1.0 wt %, preferably from about 0.05 to about 0.8 wt % and in particular from about 0.1 to about 0.5 wt % of at least one poly(C₂-C₃)alkylene glycol-modified silicone a3).
   13. The cosmetic product according to point 12, characterized in that the poly(C₂-C₃)alkylene glycol-modified silicone a3) is selected from the group of alkoxylated dimethicones, preferably from the group of ethoxylated dimethicones, and in particular from the group of compounds having the INCI name PEG-12 Dimethicone.
   14. The cosmetic product according to any of the preceding points, characterized in that the cosmetic preparation a) contains, in relation to its total weight, from about 0.1 to about 20 wt %, preferably from about 0.5 to about 15 wt % and in particular from about 1.0 to about 10 wt % of a film-forming polymer a4).
   15. The cosmetic product according to point 14, characterized in that the film-forming polymer a4) is selected from the group of anionic polymers, preferably from the group of copolymers of acrylic acid and methacrylic acid.
   16. The cosmetic product according to point 14, characterized in that the film-forming polymer a4) is selected from the group of nonionic polymers, preferably from the group of polyvinylpyrrolidones and vinylpyrrolidone//vinyl acetate copolymers, preferably polyvinylpyrrolidones.
   17. The cosmetic product according to point 14, characterized in that the film-forming polymer a4) is selected from the group of copolymers of methacryloylethyl-N,N-dimethylamine oxide.
   18. The cosmetic product according to point 14, characterized in that the film-forming polymer a4) is selected from the group of copolymers of
   i) N-tert-octylacrylamide
   ii) acrylic acid
   iii) tert-butylaminoethyl methacrylate
   iv) and optionally other monomers.
   19. The cosmetic product according to any of the preceding points, characterized in that the cosmetic preparation a) consists to a proportion of at least about 80 wt %, preferably at least about 90 wt % and in particular at least about 97 wt % of the constituents a1) and a2), in relation to its total weight.
   20. The cosmetic product according to any of the preceding points, characterized in that the cosmetic preparation a) consists to a proportion of at least about 80 wt %, preferably at least about 90 wt % and in particular at least about 97 wt % of the constituents a1), a2) and a3), in relation to its total weight.
   21. The use of a cosmetic preparation a) containing, in relation to its total weight,
   a1) from about 60 to about 98 wt % of at least one polar solvent;
   a2) from about 0.1 to about 20 wt % of at least one copolymer from the monomers
   i) N-tert-butylacrylamide
   ii) acrylic acid
   iii) ethyl acrylate
   iv) and optionally other monomers
   as process material in a device for flash evaporation.
   22. The use of a product according to any of points 1 to 20 for applying a cosmetic preparation a) to keratin-containing fibers, in particular human hair.
   23. The use of a product according to any of points 1 to 20 for the temporary shaping of keratin-containing fibers, in particular human hair.
   24. A method for the temporary shaping of keratin-containing fibers, in particular human hair, in which a cosmetic preparation a) containing, in relation to its total weight,
   a1) from about 60 to about 98 wt % of at least one polar solvent;
   a2) from about 0.1 to about 20 wt % of at least one copolymer from the monomers
   i) N-tert-butylacrylamide
   ii) acrylic acid
   iii) ethyl acrylate
   iv) and optionally other monomers
   is applied to the keratin-containing fibers by employing a device for flash evaporation.
   25. The method according to point 24, characterized in that,
from a storage container in the interior of which a pressure corresponding to the ambient pressure prevails, a partial quantity of the cosmetic preparation a) located in this storage container is transferred into a container b1);
then the access between the storage container and the container b1) is interrupted by a flow control component, such that the flow of the cosmetic preparation a) from the storage container into the container b1) can be interrupted;
then the cosmetic preparation a) located in the container b1) which is sealed off from the environment is heated by employing a heating device so that the pressure in the interior of the container b1) increases to values above the ambient pressure, preferably to values between about 1.1 and about 8 bar, in particular to values between about 1.2 and about 4 bar;
then the container b1) which is at a pressure above the ambient pressure is opened in such a way as to enable at least a partial quantity, preferably at least about 50 wt %, more preferably at least about 80 wt % and in particular at least about 90 wt %, of the cosmetic preparation located in the container b1) to be released from the container b1) into the environment, thereby reducing the pressure prevailing in the container b1) at the time of opening of the container.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A cosmetic product comprising:
a) a cosmetic preparation comprising, in relation to its total weight,
a1) from about 60 to about 98 wt % of at least one polar solvent; and
a2) from about 0.1 to about 20 wt % of at least one copolymer from the monomers;
i) N-tert-butylacrylamide,
ii) acrylic acid,
iii) ethyl acrylate, and
iv) optionally other monomers; and
b) a device for flash evaporation of the cosmetic preparation a).

2. The cosmetic product according to claim 1, wherein the device for flash evaporation comprises a container b1) and a heating device b2) and is designed such that:
the cosmetic preparation a) can be accommodated in the interior of the container b1),
the interior of the container b1) at least partially filled with the cosmetic preparation a) can be closed,
the cosmetic preparation a) in the closed interior of the container b1) can be heated by employing the heating device b2), the pressure thereby being increased, and
the heated cosmetic preparation a) can be released from the interior of the container b1) into the environment, the pressure thereby being reduced.

3. The cosmetic product according to claim 1, wherein the proportion by weight of the polar solvent a1) in relation to the total weight of the cosmetic preparation a) is from about 70 to about 98 wt %.

4. The cosmetic product according to claim 1, wherein the polar solvent a1) has a boiling point (20° C., 1013 mbar) between about 50 and about 110° C.

5. The cosmetic product according to claim 1, wherein the polar solvent a1) is selected from the group of ethanol, isopropanol, and combinations thereof.

6. The cosmetic product according to claim 1, wherein the proportion by weight of the copolymer a2) in relation to the total weight of the cosmetic preparation a) is from about 0.5 to about 15 wt %.

7. The cosmetic product according to claim 1, wherein the cosmetic preparation a) comprises, in relation to its total weight, from about 0.01 to about 1.0 wt % of at least one poly(C₂-C₃)alkylene glycol-modified silicone a3).

8. A cosmetic preparation a) comprising, in relation to its total weight,
a1) from about 60 to about 98 wt % of at least one polar solvent;
a2) from about 0.1 to about 20 wt % of at least one copolymer from the monomers; and
i) N-tert-butylacrylamide,
ii) acrylic acid,
iii) ethyl acrylate, and
iv) optionally other monomers,
wherein the cosmetic preparation a) is utilized as a material in a device for flash evaporation.

9. The cosmetic product according to claim 1, wherein the cosmetic product is utilized for applying the cosmetic preparation a) to keratin-containing fibers.

10. The cosmetic product according to claim 1, wherein the cosmetic product is utilized for the temporary shaping of keratin-containing fibers.

11. A method for the temporary shaping of keratin-containing fibers, the method comprising applying a cosmetic preparation a) to the keratin-containing fibers by employing a device for flash evaporation, the cosmetic preparation a) comprising, in relation to its total weight,
a1) from about 60 to about 98 wt % of at least one polar solvent; and
a2) from about 0.1 to about 20 wt % of at least one copolymer from the monomers;
i) N-tert-butylacrylamide,
ii) acrylic acid,
iii) ethyl acrylate, and
iv) optionally other monomers.

12. The method according to claim 11, wherein,
from a storage container in the interior of which a pressure corresponding to the ambient pressure prevails, a partial quantity of the cosmetic preparation a) located in this storage container is transferred into a container b1);
then the access between the storage container and the container b1) is interrupted by a flow control component, such that the flow of the cosmetic preparation a) from the storage container into the container b1) can be interrupted;
then the cosmetic preparation a) located in the container b1) which is sealed off from the environment is heated by employing a heating device so that the pressure in the interior of the container b 1) increases to values above the ambient pressure;
then the container b1) which is at a pressure above the ambient pressure is opened in such a way as to enable at least a partial quantity of the cosmetic preparation located in the container b1) to be released from the container b1) into the environment, thereby reducing the pressure prevailing in the container b1) at the time of opening of the container.

13. The cosmetic product according to claim 7, wherein the poly(C₂-C₃)alkylene glycol-modified silicone a3) is selected from the group of alkoxylated dimethicones and combinations thereof.

14. The cosmetic product according to claim 13, wherein the poly(C₂-C₃)alkylene glycol-modified silicone a3) is selected from the group of compounds having the INCI name PEG-12 Dimethicone and combinations thereof.

15. The cosmetic product according to claim 1, wherein the cosmetic preparation a) contains, in relation to its total weight, from about 0.1 to about 20 wt % of a film-forming polymer a4).

16. The cosmetic product according to claim 15, wherein the film-forming polymer a4) is selected from the group of anionic polymers and combinations thereof.

17. The cosmetic product according to claim 15, wherein the film-forming polymer a4) is selected from the group of nonionic polymers and combinations thereof.

18. The cosmetic product according to claim 15, wherein the film-forming polymer a4) is selected from the group of copolymers of methacryloylethyl-N,N-dimethylamine oxide and combinations thereof.

19. The cosmetic product according to claim 15, wherein the film-forming polymer a4) is selected from the group of copolymers of:
i) N-tert-octylacrylamide;
ii) acrylic acid;
iii) tert-butylaminoethyl methacrylate; and
iv) and optionally other monomers.

20. The cosmetic product according to claim 1, wherein the cosmetic preparation a) comprises of a proportion of at least about 80 wt % of the constituents a1) and a2), in relation to its total weight.
